(19) United States
(12) Patent Application Publication
(10) Pub. No.: US 2017/0095199 A1
(43) Pub. Date: Apr. 6, 2017

(54) BIOSIGNAL MEASUREMENT, ANALYSIS AND NEUROSTIMULATION

(71) Applicant: V1bes, Inc., Palo Alto, CA (US)

(72) Inventor: Gustaf KRANCK, San Jose, CA (US)

(21) Appl. No.: 15/282,332

(22) Filed: Sep. 30, 2016

Related U.S. Application Data
(60) Provisional application No. 62/235,812, filed on Oct. 1, 2015.

(51) Int. Cl. A61B 5/00; A61N 2/02; A61B 5/0476; A61N 2/00; A61B 5/04; A61B 5/024

(52) U.S. Cl. CPC A61B 5/4836; A61B 5/04005; A61B 5/02438; A61B 5/0476; A61N 2/006; A61N 2/02

(57) ABSTRACT

Systems and methods here include measuring and analyzing biosignals. Some embodiments include measuring a magnetic bio signal using a coiled wire system, sending the measured bio signal to a computer for processing, receiving the measured bio signal and processing the bio signal, choosing a stimulation signal, based on the received measured bio signal, sending the chosen stimulation signal to a stimulation device, and administering the stimulation signal with the stimulation device.

### CROSS REFERENCE

This application relates to and claims priority from U.S. Provisional Application 62/235,812 filed on 1 Oct. 2015, the entirety is hereby incorporated by reference.

### TECHNICAL FIELD

This application relates to the field of human biosignal measurement, analytics methods and vagus nerve stimulation by using non-invasive magnetic fields.

### BACKGROUND

Stress is a major health problem in today's society. Humans are constantly trying to find means to relax their brains and alter brain states for the better. At the same time modern society demands increased cognition capacity due to the quickly increasing information flows and complexity of life. The brain has not been able to adapt itself and hence people feel a strong need to find new ways to relax or improve cognition and energy levels. As society is becoming increasingly technology-driven, society is ready and in need of technical solutions for how to control mental states.

Humans have always strived to find conscious means to alter mind states. Alcohol, mushrooms, herbs, smoking, music and meditation are all examples of traditional mind state altering methods.

The latest technological development has enabled the use of wireless, very advanced measurement electronics which much better than before can detect subtle changes in the human body's biosignals. This data and latest processing capacities in smartphones and computers have enabled new discoveries and innovations on how to use biosignals and new ways to do neurostimulation.

Biosignals as used herein include biological signals in a living being which may be monitored, such as Electroencephalogram (EEG), Electrocardiogram (ECG), Electromyogram (EMG), and/or Galvanic skin response (GSR).

Traditionally brainwave measurements (EEG) have relied on the ear or the skull for ‘grounding’ of the signal in order to discover the electrical activity at the point of the measurement electrode. Because processing power and measurement precision has improved considerably during past years, the inventor discovered that it is possible to use the finger as ground when using a single point electrode on the forehead and still obtain valid and informative data from the brain and the body's biosignals.

Heart rate variability (HRV) is currently the only widely used method to evaluate the heart stress levels. HRV has a major statistically originating flaw when used in short term measurements. Since the heart beats only on average once per second—the statistical accuracy when measuring only 20 or 30 heartbeats (25 second test), is on average low. There is great demand for quick and reliable new ways to determine the stress level of the heart. This invention is in part targeting this problem.

Vagus Nerve Stimulation (VNS) is a common medical treatment method for various neurological disorders and diseases. The main VNS method is implanting electrical stimulation electronics that are situated around the vagus nerve at the throat area. These have a separate battery and signal generation unit which is implanted in the chest area. These types of implants are most commonly used for treating epilepsy. There is much research in the field and there are also non-invasive medical devices that induce electrical current on the surface of the skin above the throat area (for instance Electrocore Medical LLC VNS devices).

### SUMMARY

The present invention provides methods, apparatus and analytical means to measure and record human biosignals and selectively apply magnetic field stimulation to the nervous system to achieve a desired result.

Systems and methods here include measuring and analyzing biosignals. Some embodiments include measuring a magnetic bio signal using a coiled wire system, sending the measured bio signal to a computer for processing, receiving the measured bio signal and processing the bio signal, choosing a stimulation signal, based on the received measured bio signal, sending the chosen stimulation signal to a stimulation device, and administering the stimulation signal with the stimulation device.

Systems and methods include, in some embodiments used for analyzing biosignals, including a dual purpose measuring/stimulation device, in communication with a smartphone running a software application, a back end server, and a data storage, the measuring/stimulation device configured to, measure electromagnetic fields of a user; send the measured electromagnetic fields to the smartphone software application; the software application configured to, send the received data to the back end server for processing; receive processed data from the back end server; send a stimulation signal to the measuring/stimulation device according to the received processed data. In some embodiments, the stimulation signal is derived from recorded user electromagnetic fields. In some embodiments, the stimulation uses low strength magnetic field between 0.1 and 6 micro Teslas. In some embodiments, the stimulation device is stereo with two coils which work in concert. In some embodiments, the measuring/stimulation device is further configured to play the stimulation signal. In some embodiments, the device includes wire coils in a headset arrangement. In some embodiments, the bio signal is a heart rate. In some embodiments, the bio signal is a brainwave.

### BRIEF DESCRIPTION OF THE FIGURES

FIG. 1 is a diagram showing an example network configuration according to certain embodiments herein.

FIG. 2A is a diagram showing an example measuring device according to certain embodiments herein.

FIG. 2B is a diagram showing another example measuring device according to certain embodiments herein.

FIG. 3 is a diagram showing an example stimulation device on a user according to certain embodiments herein.

FIG. 4 is a diagram showing an example stimulation device according to certain embodiments herein.

FIG. 5 is a flow chart showing example steps taken to practice certain embodiments herein.

FIG. 6 is a diagram showing example graphical user interfaces according to certain embodiments herein.

FIG. 7 is a diagram showing more example graphical user interfaces according to certain embodiments herein.

FIG. 8 is a diagram showing more example graphical user interfaces according to certain embodiments herein.

FIG. 9 is a chart showing example analytic analysis on data gathered from certain embodiments herein.

FIG. 10 is a chart showing another example analytic analysis on data gathered from certain embodiments herein.

FIG. 11 is a chart showing another example analytic analysis on data gathered from certain embodiments herein.

FIG. 12 is a chart showing another example analytic analysis on data gathered from certain embodiments herein.

FIG. 13 is a diagram of an example hardware computing device which may be used to practice the various embodiments described herein.

FIG. 14 is a screenshot of a social networking example according to certain embodiments herein.

### DETAILED DESCRIPTION

Reference will now be made in detail to embodiments, examples of which are illustrated in the accompanying drawings. In the following detailed description, numerous specific details are set forth in order to provide a sufficient understanding of the subject matter presented herein. But it will be apparent to one of ordinary skill in the art that the subject matter may be practiced without these specific details. Moreover, the particular embodiments described herein are provided by way of example and should not be used to limit the scope of the invention to these particular embodiments. In other instances, well-known data structures, timing protocols, software operations, procedures, and components have not been described in detail so as not to unnecessarily obscure aspects of the embodiments of the invention.

### Overview

The use of magnetic field in brain stimulation may produce various responses by the human body, depending on what the stimulation is and where it is applied. For example, low energy magnetic stimulation of the vagus nerve may relax the brain. Stimulation may excite the brain. Similarly, the heart may be excited or relaxed. And although the correlation between brain activity and heart activity are not always one-to-one, stimulation may be used to achieve various goals.

The purpose of stimulation is to help the user to relax, improve cognition, become more alert (energetic), or other general or specific effects on the brain and/or nervous system, such as lowering blood pressure.

For example, it may be relaxing to the brain to run, but excite the heart. Similarly an alcoholic drink may relax the brain but excite the heart. Certain music may excite the brain but relax the heart. Each of these may be different for different people, but by gathering data and analyzing it for individual people, certain stimulations could be used to achieve various goals.

In certain examples, the stimulation itself could be a low energy electromagnetic signal applied to the vagus nerve area of the neck. Such signal could be any of various signal patterns, which may be used to achieve any of various goals. Signal patterns may be a frequency or tone. Signal patterns may be a music track. And some signal patters may be created from recording biological events, such as brain waves themselves. Stimulation with human biosignal patterns may be more efficient at stimulation than the use of mechanically and non-human origin signal patterns and programs. Research implies that the body's natural ‘neural-defense’ against external electrical or magnetic fields can in part be lowered with the use of body-originated biosignal recordings as the basis for stimulation programs.

By using various devices as described herein, collecting data from the user, and applying various algorithms to the data, vagus nerve stimulation may be conducted, sometimes with the use of collected brain wave patterns from the user herself. Embodiments may include use of various pieces of hardware, such as a measurement device, and a stimulation device, working with a software application on a mobile smartphone connected via a network to a back end analytics and data storage center.

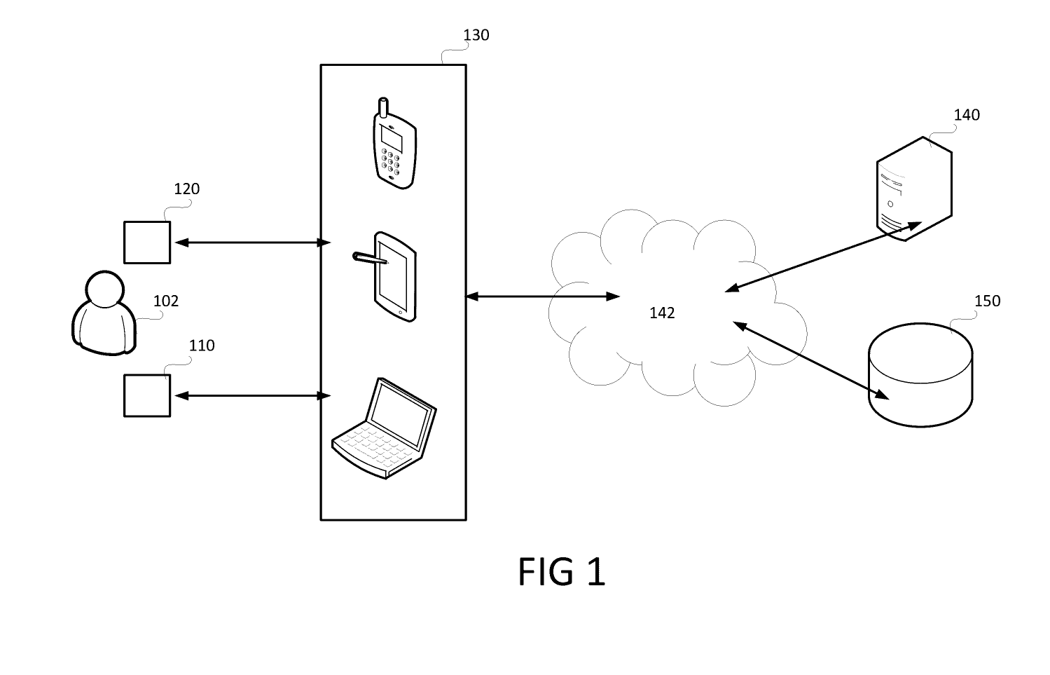

FIG. 1 shows an example network diagram of these components. For example, the measurement device 110 could be any kind of biosignal measurement device that could be used for electro cardio gram (ECG/EKG), and/or electroencephalogram (EEG). Such a measurement device 110 could be in the form of a portable ring, in certain embodiments, as described here. In certain examples, it includes a wireless transmitter in order to send data measured from the user 102, to a mobile device running a software application 130, and thereby to a back end analytics system 140 and data storage 150 through a network 142.

In certain embodiments, another piece of hardware, a stimulator 120 could be used in conjunction with the other systems. Such a stimulator 120 could be a device that creates magnetic field impulses through coils of wire as described herein. Such a stimulator 120 could include a wireless transmitter in order to receive data from the back end systems 140 and data storage 150 through the mobile device software application 130. In this way, the stimulator 120 could receive the data such as a music file format and play the music file format, not as audio, but as magnetic impulses through coiled wires.

In such an example, the back end systems 140 could receive the measurement data from the measurement device 110, analyze the data, and either send the stimulator 120 a data file or present the user various options of data files to play, depending on the goal of the stimulation. The systems and methods described here are primarily to be used as a lifestyle device.

### Measurement Device Examples

In certain examples, a measurement device may be used to gather data about a user. The device may measure brainwave and/or electroencephalogram (EEG). The device may also be used to measure the heart's electro cardio gram (ECG/EKG). As these measurements are of electromagnetic signals emitted by the brain and heart respectively, the same device can be used. In certain examples, the measurement device includes a loop so it may be worn as a ring by a user. And certain examples also include wireless transmission so the measure data may be sent to a smartphone device or any other device which may retransmit or store the measured data, as described herein.

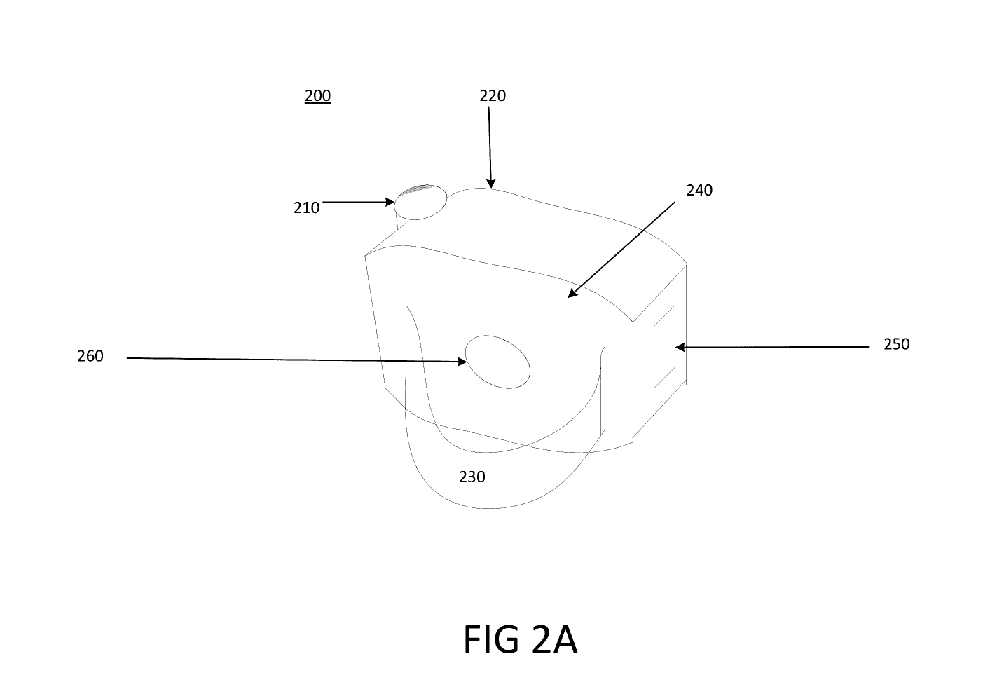

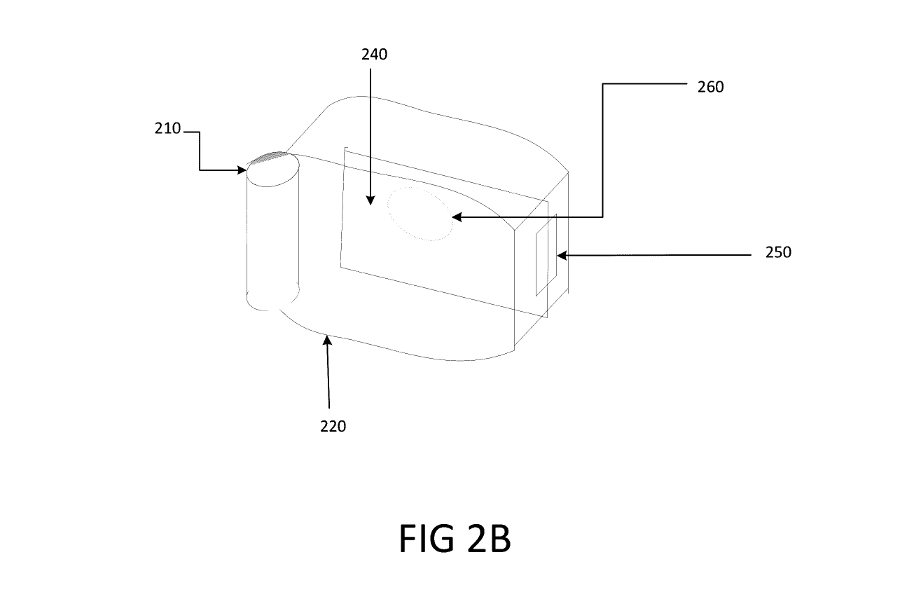

FIG. 2A and FIG. 2B shows the various components of an example measurement device 200. The measurement device includes a conductive ring 210, used to receive the biosignals from the user. These signals may be from either the brain or heart, by placing the conductive ring on either the thumb for the heart or the forehead for the brain. Any of other various placements may be used.

The measurement device example also includes a cover 220, strap or flexible band 230 to fit on the finger, measurement electronics 240, battery unit, data and charging connector port 250, electrode which is in contact with the finger surface skin 260, pipe electrode that in contact with forehead 210, second hand finger or free in air acting as EMF antenna.

The technical specifications generally used in the measurement electronics include 2-electrode (bipolar) signal acquisition, 2000X high gain amplifier. Also 256 Hz or 512 Hz sampling rate, 13 bit effective dynamic range. A Built-in 3D (XYZ) accelerometer. Rechargeable LIR1220 button cell, typically >16 hrs use time with full charge. Bio-signal recording of 11+hours on sensor unit memory module. In certain examples, one silver coated dry electrode touching skin for continuous body-measurement. In certain examples, one silver pipe electrode as touch electrode for EEG, ECG and EMG and EMF.

The measurement electronics of measurement device is configured to measure biosignal activity by detecting very subtle voltage fluctuations between the sensors skin electrode and pipe electrode. The measured voltage fluctuations are caused by current flows within the neurons of the brain (Electroencephalography EEG), electrical activity of the heart (Electrocardiograph, ECG or EKG), electrical activity of muscles (Electromyography, EMG), electrical conductance of the skin which varies depending on the amount of sweat-induced moisture on the skin (Skin Conductance) or electromagnetic fields in the surrounding of the body. The measurement device and stimulation device do not induce any electrical- or other impulses into the human body.

The sensors may use low power wireless signals to transmit data to the smartphone, other type of receive or computer. Such a wireless connection could be any of various things including Bluetooth Low Energy, ZigBee, WiFi, cellular or any other kind of wireless transmission.

The primary use case for measurement device is to measure Brainwaves (EEG). This is done so that one measurement point (silver electrode under the device) is touching the skin on the finger and the other electrode is touching (silver electrode-pipe) the forehead. The user will hold the device to the forehead while hands are as relaxed as possible, mainly so that the elbow is resting on a table while doing the measurement. The finger acts as the second measurement point for the electrical measurement, also in normal EEG measurement called the grounding point. In traditional EEG measurement devices the ground is usually on the ear or at some other points on the head. The device is uses the finger surface as grounding point. The measurement device may then transfer the measurement data in real time to the smartphone for the application or any other computer.

Another ability of the measurement device is to measure and collect data on the electrical heart rate when the user places a finger or thumb on top of the silver electrode pipe. The measurement position when doing ECG measurements are normally so that the user holds the hands crossed on a table in front of him or her.

Another use area for the same measurement device is that it can measure vagus nerve biological signals when the user holds it on the throat above the vagus nerve while the measurement electrode touches the skin.

Another use area for the same measurement device is that it can measure surrounding electromagnetic fields (EMF) by holding the hand in the air. The measurement point then acts as an antenna and the finger as ground. Such a measurement may be used by the back end to calibrate the user data, and remove any background noise or interference which is measured in the air and surrounding environment.

In certain examples, a measured EEG and an EKG can be combined to analyze blood pressure in a user.

### Stimulation Device Examples

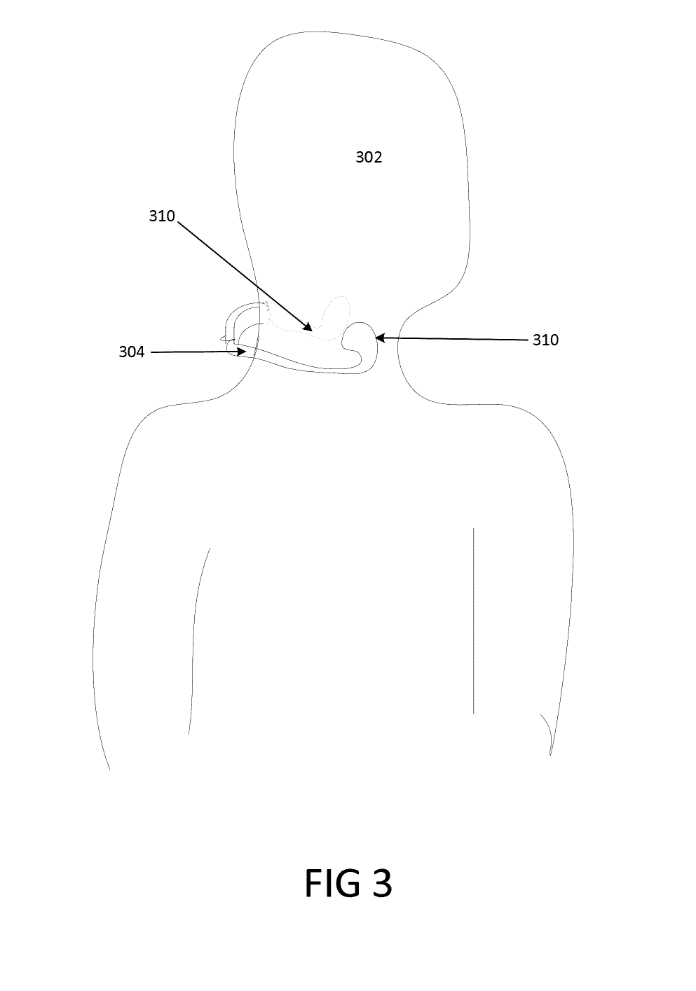

In certain examples, not only is user biosignal data measured and stored, but a stimulation device may be used to send biosignals back into the body. The signals sent into the body may be through any of various points in the body including the neck area where the vagus nerve runs. FIG. 3 shows an example user 302 and the two sides of the neck 310 where the vagus nerve runs. Thus, instead of stimulating just one area, the stimulation device here could be used in stereo, to stimulate both sides of the neck and the two vagus nerve areas. Such neurostimulation may be done through a non-invasive vagus nerve stimulation device 304 which uses dynamic time-varying magnetic fields applied on the throat above the vagus nerve (VNMS).

Generation of electromagnetic signals may be accomplished with the stimulation device. The magnetic fields may be generated by sending time varying dynamic electrical currents to the left and right coils 310 in the stimulation device 304. To achieve the intended stimulation effect, the electric current thereby locally vary to each coil both in strength or frequency pattern and the magnetic fields therefore are most usually time varying and dynamically changing between left and right sides. The increased stimulation effect arises in part from this simultaneous but at different strengths and frequencies generated signal patterns that fluctuate between the left and right side vagus nerve. The stimulation device may use any arrangement of coils on both left and right side to generate the magnetic fields.

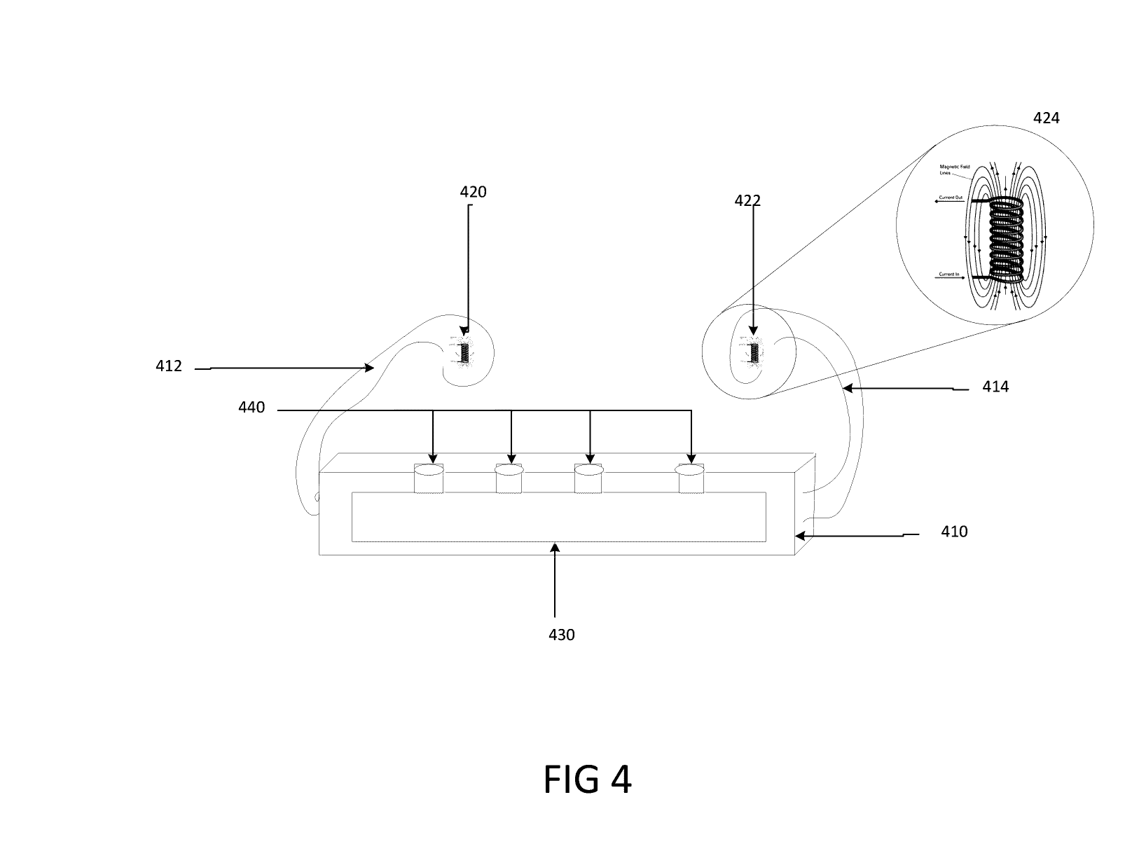

FIG. 4 shows an example stimulation device and its component parts. FIG. 4 shows a housing for the electronics 410, an adjustable left 412 and right 414 extensions, an on/off button, volume adjustment buttons, left 420 and right 422 coils for the creation of magnetic fields. An example detail of a magnetic coil 424 is shown as well.

Not shown are a possible plastic flexible strap to improve the fit on the neck. Inside the cover 410 are the electronics 430 for receiving the signal data and the generation of the signal, charging unit and battery unit with data- and charging connector port 440, connection ports for input and output of signals, an antenna to communicate via any kind of wireless system with the smartphone and application. The stimulation device may receive the stimulation programs through a Bluetooth low energy signal protocol ranging between 2400 and 2500 MHz from the smartphone or other wireless transmitter.

The stimulation device can be used to play any of various kinds of stimulation signals, through the coils. The stimulation device could be used to apply low energy electromagnetic signals to the human body to achieve any of various results such as relaxation or excitement. The signal patterns used to create such results could be created from any source such as a computer generated tone, a music track, a brainwave signal recording, a vagus nerve biosignal recording, or a combination of any of these.

It should be noted that signals which are derived from or recorded from biological sources, may have improved stimulation effects. Thus, signals from recordings of brain waves, vagus nerve waves, or other biological signals, may be recorded and used as signals for the stimulation itself. The recorded biosignals, as recorded with measurement device, are adapted and transformed so that they achieve maximum stimulation effect.

Additionally or alternatively, the stimulation device could include a standard earphone or speaker set. Thus, the stimulation device could provide simultaneous audio signals and optionally low energy electric pulsation on the surface of the outer ear channel with the Stimulation device's earphones.

In certain examples, the stimulation can be used to lower blood pressure in a user.

### Example Flow Diagram

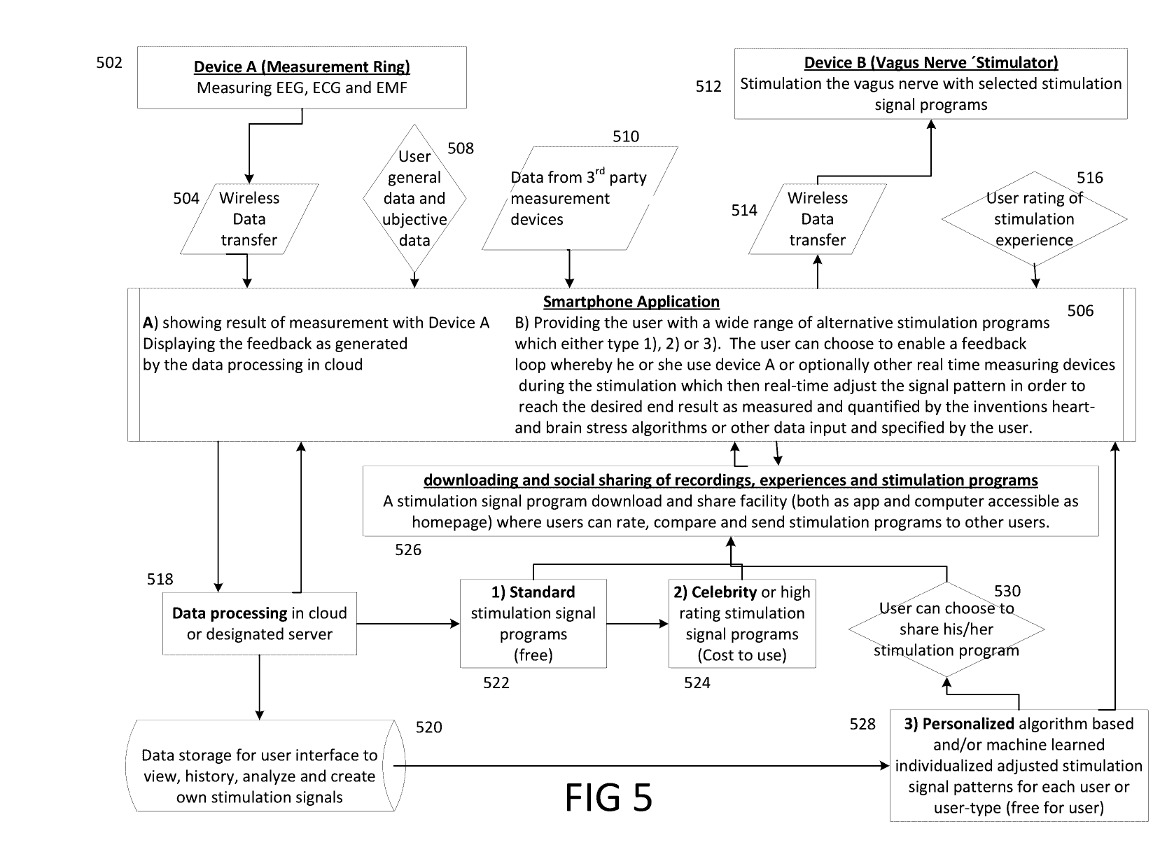

FIG. 5 shows a flow chart diagram that explains example steps of how the system may carry out certain aspects of the disclosure here. FIG. 5 begins with the measurement device 502 which could be the ring in certain examples. The measurement device is coupled wirelessly 504 to a smartphone or other computing device hosting an application 506 which can interface with the measuring device 502. The smartphone application can perform as described herein, instructing the user how to use the measuring device, receiving and storing data received from it, displaying any of various data to the user, etc. The application may also receive user generated data 508 from the user herself. The application may receive data from third party measuring devices of any sort 510. The other device which may be used is a stimulation device 512 which may also be in communication with the smartphone 506 via a wireless connection 514. The smartphone application 506 may also prompt the user to input rating of their experience 516 and store that data as well.

FIG. 5 also shows back end analytics and data storage steps. As shown as an example, the smartphone application 506 may send data to be processed 518 to a distributed data processing or local data processor. Such analyzed data results may be sent back to the application 506 or sent to data storage 520 which could be central, local, and/or distributed data storage.

The data processing 518 may also determine what kind of signal to send to the user in a particular session. Such a determination may be for a particular stimulation program that is offered free to a user 522 or at a cost for a celebrity or other endorsement 524. The systems can then allow the user to download and share information 526 which could access the smartphone application 506 for social networking attributes, accounts, sharing information, etc.

Additionally or alternatively, the data 520 may be used to create personalized algorithms which are products of machine learning to customize signal patterns, make recommendations, adjust settings, etc. for a particular user 528. Such personalized information can also be shared by a user 530 again through the smartphone application 506 and related aspects.

### Stimulation Program Examples

In certain examples the signals that are used in the stimulation device are generated by the back end systems, sent to the smartphone application and from there to the stimulation device for playing. The signals are stored and played similar to how music files are stored and played by a music service. These files can be stored or sent to a smartphone, and from there to a Bluetooth wireless headset. But here, instead of speakers, two coils create an electro-magnetic signal to stimulate the vagus nerves in the neck.

The signals which could be used to stimulate the body could be any of various signals. In some examples, the networked data storage can store and have sent, stimulation programs which are generated from the own user's stress test, EEG recordings or vagus nerve recordings done with the measurement device. This may be done so that a 20-60 seconds long recording may be repeated to extend to mainly 5, 10, 15 or 30 minutes long stimulation programs. The recordings may be copied after each other with optional changes in time-varying strength-, right/left balance and equalization adjustment of frequencies or frequency areas and their strengths.

The stimulation device causes electrical stimulation currents in the body tissue and vagus according to the principle of electromagnetic induction for stimulating live tissue and neurons. The system primarily uses Waveform Audio File Format or more commonly known as way-format extension. An MP3 could be used as well. The stimulation is done with frequencies ranging from 0.1 Hz to maximum 20,000 Hz, the main frequency area being between 0.1 and 150 Hz.

In certain embodiments, the user can determine in the application and networked data storage which type of stimulation program he or she wants to generate. Example stimulation program types include but are not limited to, relaxing, energizing and cognitive enhancing. The stimulation programs can also be classified according to sub-groups which determine whether the said stimulation program can influence sleep, induce sleep, help to awake, help digestion, decrease the feeling of hunger, increase or decrease attention and create various types of sensations in the body or head.

The stimulation program type can also be based on the classification of signal patterns together with subjective experiences reported by the users in the social sharing site.

The main type-classification of recorded brainwave- and heart rate biosignals may be based on a user's Heart Frequency Stress Index (HFSI) and Brain Stress Index (BSI) parameters. If the stress index show a low stress level, then the recorded signal may be suitable for using in relaxation stimulation programs. If the stress indexes are high and hence indicate high stress in the heart and/or brain, then the signals can be used in energizing and/or cognition enhancing stimulation programs. The relation between heart- and brain stress indexes is also one potential determinant for the suitability of recordings in different types of stimulation programs.

In addition to using stress indexes, the classifications can also additionally be based on parameters from high/peak/bottom/low frequency pattern recognition, subjective feedback and/or settings determined by the machine learning individual algorithms applicable to the user.

In example embodiments for creating stimulation programs, in addition to using recorded brainwave patterns, the stimulation programs can use fixed frequency patterns based on research and the machine learning individual algorithms applicable to the user. These frequency patterns can for instance be time-varying dynamic frequencies such as 8-11 Hz, 14-17 Hz, 19-22 Hz, 25-29 Hz, 32-35 Hz, 39-42 Hz, 63-67 Hz or 85-95 Hz. In certain embodiments, stimulation program generation, additional fixed frequencies or groups of frequencies may be inserted into the stimulation programs.

In certain example embodiments of creating stimulation programs, an automatic algorithm may change and adjust the stimulation program during stimulation. This pattern change may be done based on the real-time recording of brainwave signals which are done while the stimulation is ongoing. This type of stimulation program may be referred to as real-time dynamic vagus stimulation. For example, when machine learning has determined the most common brainwave pattern of the user when he or she is alert and energetic, then the dynamic vagus stimulation function may aim to reach similar brainwave pattern during stimulation. This goal may be reached by a scanning pattern in the stimulation program in order to recognize which stimulation pattern is most suitable for reaching the intended end brainwave pattern of the user. This dynamic function can be real time continuous and may be applied for the whole stimulation program in certain examples.

In certain example embodiments, music may be used as a stimulation program. The music file is sent to the stimulation device as if the stimulation device is a headphone or audio speaker, but instead a magnetic field is generated instead of vibrating a paper speaker. The user can have a different kind of phenomenological conscious experience of the music than with usual musical reproduction devices through audio alone. The user may also feel various kinds of sensations in the head, throat or body due to the vagus nerve stimulation, which he or she otherwise would not experience. The vagus stimulation can therefore give new types of experiences from music.

Certain example embodiments can save subjective feedback from the user before and after he or she has used a stimulation program. This feedback can also be as 1 to 5 star rating of the quality of the stimulation experience such as the effectiveness, strength, pleasantness, relaxing effect and/or energizing effect. In an example, when the user chooses a suitable stimulation program, these start-ratings can be on selection criteria in the application.

Certain example embodiments can also use music as stimulation program either without alteration or mixed with recorded biosignals or machine generated signals to achieve different stimulation effects. For example, a brain wave signal from a celebrity may be combined with a song that the celebrity performs. Such a combination of music and biological signals may have certain effects to a user of the stimulator device or may be desired by fans of celebrities.

The stimulation with the stimulation device, the application and networked storage may all work simultaneously as the user is doing real time wireless measurements with measurement device. When using real time feedback, the stimulation signals can auto-adjust and also create new signals in real time, thereby achieving better stimulation effects.

The stimulation device may have a time-varying magnetic field strength of 0.001 to 3 micro Tesla. This falls below the internationally a recognized reference level for the general public when being exposed to time-varying magnetic field is considered 6.25 micro Tesla.

In certain example embodiments, user experience can be further enhanced when the user at the same time as experiencing magnetic stimulation of the throat vagus nerve, use earphones which are connected to the stimulation device body-part and simultaneously with the stimulation, replay to the ear the same stimulation program or music. These earphones can either be standard earphones with insulation plastic parts or then the inventors designed earphones where the plastic part touching the ear channel is partially electrically conductive or contains a coil generating magnetic fields. The earphones are built so that the ear can with these conductive earphones, receive very low electric impulses or magnetic fields as part of the stimulation and hence activate the part of vagus nerve which is located by the ear channel. This part of the innovation may include tinnitus vagus nerve ear electric pulse stimulation devices in an addition to throat vagal nerve magnetic field stimulation by stimulation device. By combining magnetic field stimulation and weak electric pulse stimulation in the ear, the stimulation device stimulation effect may be improved.

### Mobile Device Application

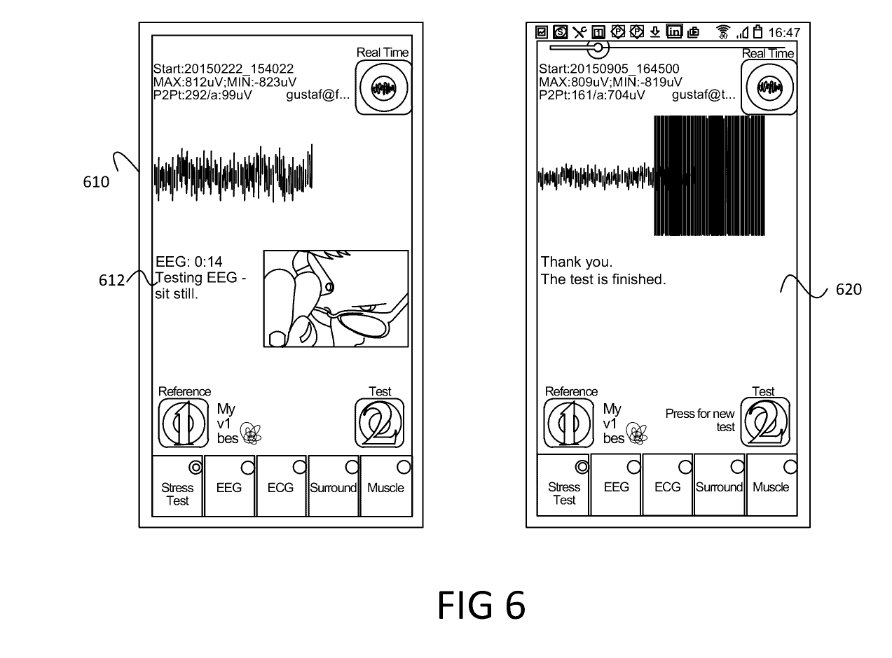

In FIG. 6 an example smartphone software application graphical user interface (GUI) is shown. In this example, the application is used to communicate with both the measurement device to receive biometric data and the stimulation device to send data files to play. The application is also used to communicate with the back end servers and data storage through whichever wireless connection the particular smartphone is implementing at any given time. For example, the particular smartphone could utilize a cellular connection or a WiFi connection to the Internet. The back end systems and data storage also communicate via the Internet or other various computer networks, and may communicate with the software application running on the mobile device smartphone. The smartphone software application may also be used to instruct the user as to how and when to use the measurement device and/or stimulation device.

Through this mobile device smartphone application, the overall systems can collect data from the measurement device, send the data, along with other gathered information about the particular user, to the back end systems. At the back end systems, the servers could apply any of various algorithms to the data, analyze the data, cause storage of the data and analytics in the data storage, and determine which kind of data file should be played by the stimulation device for the user. In certain examples, the back end system may provide a menu of options to the user via the mobile smartphone software application. In certain examples, the data files for stimulation re stored in the back end storage, and in some examples, the data files for stimulation are stored locally in the smartphone.

The application running on the smartphone may also be used to gather information about the user, such as name, age, weight, height, along with social information such as whether they smoke, feel depressed, and whether they wish to be excited or relaxed by the stimulation. The application running on the smartphone may also be used to provide feedback to the user including charts, graphs, data plots and recommendations after their data has been analyzed.

FIG. 6 shows an example screenshot of a GUI instructing a user how to hold the measurement device to his head 610 and a display of the recorded brainwave activity 612. The second GUI shows that the application has recorded all of the data it needs for this test, and instructs the user to remove the device and that the test is finished 620.

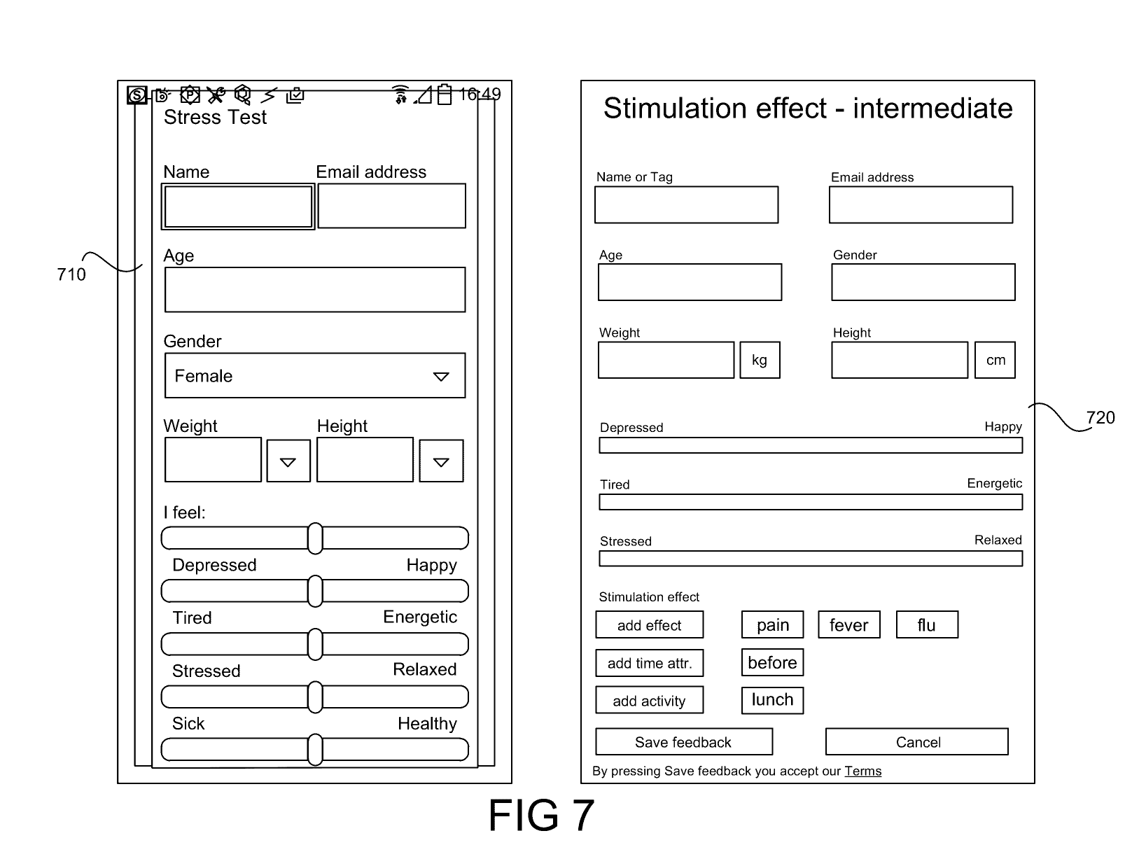

FIG. 7 shows two GUIs of the application which can be used for collecting information about user 710. Another GUI shows a screenshot of the application which, before sending the recorded data to the networked storage, the application may provide the user input fields for subjective feedback and individual data 720. In the subjective feedback, the user may describe different mind-state and health features such as for example age, weight, height, feelings, health, timing, previous activity and dietary data. This data can be used to classify the recordings for later use in brainwave stimulation programs.

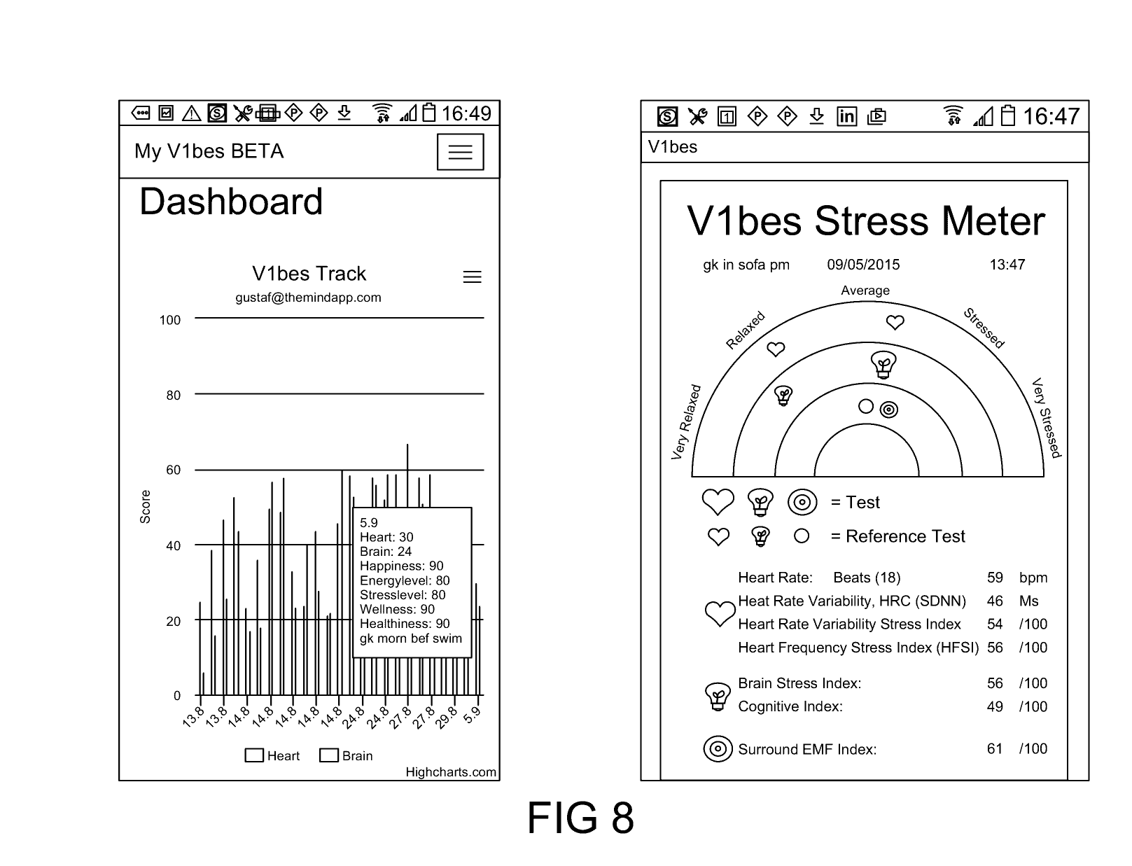

FIG. 8 is a diagram showing an example GUI used to show users a dashboard 810 of data gathered from them. FIG. 8 also shows a GUI of measured brainwave (EEG) and heart rate (ECG) measurements as presented to the user as Stress Meter 820. The application stress test program may automatically first record heart rate for 20-60 seconds (ECG), then brainwaves for 20-60 seconds (EEG) and finally also the electromagnetic surroundings by keeping the sensor in the air for 3-10 seconds. The recorded data may be sent to the networked data storage automatically where it can be analyzed and feedback information sent to the user and data then stored in the users own data-depository.

In certain examples, the EEG recordings may be analyzed and presented to the user as a ‘Brain Stress Index’ which is giving a scale from 0-100 where 0 is the least stressed and 100 is the most stressed. In FIG. 8, the stress information is visualized on a chart 820 which may help users understand their status and the data better. All of this information may be packaged and presented to the user via the application, or sent to the user as an email, SMS or other communication.

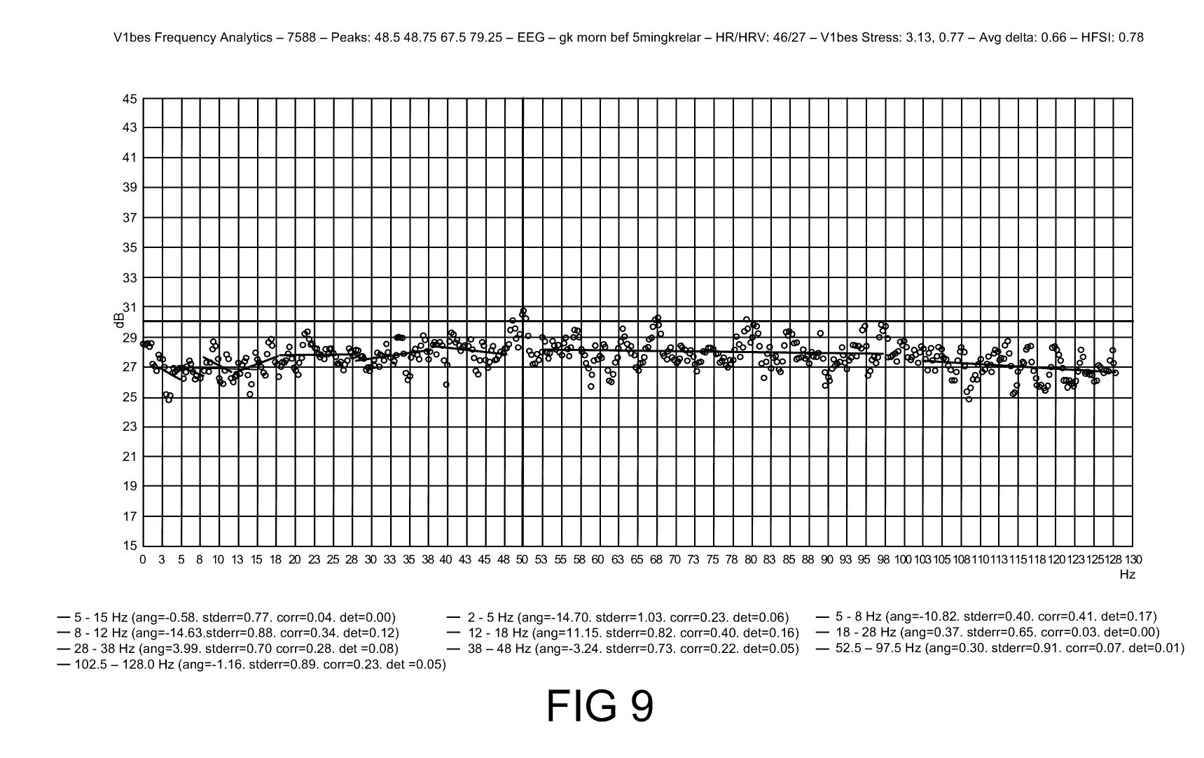

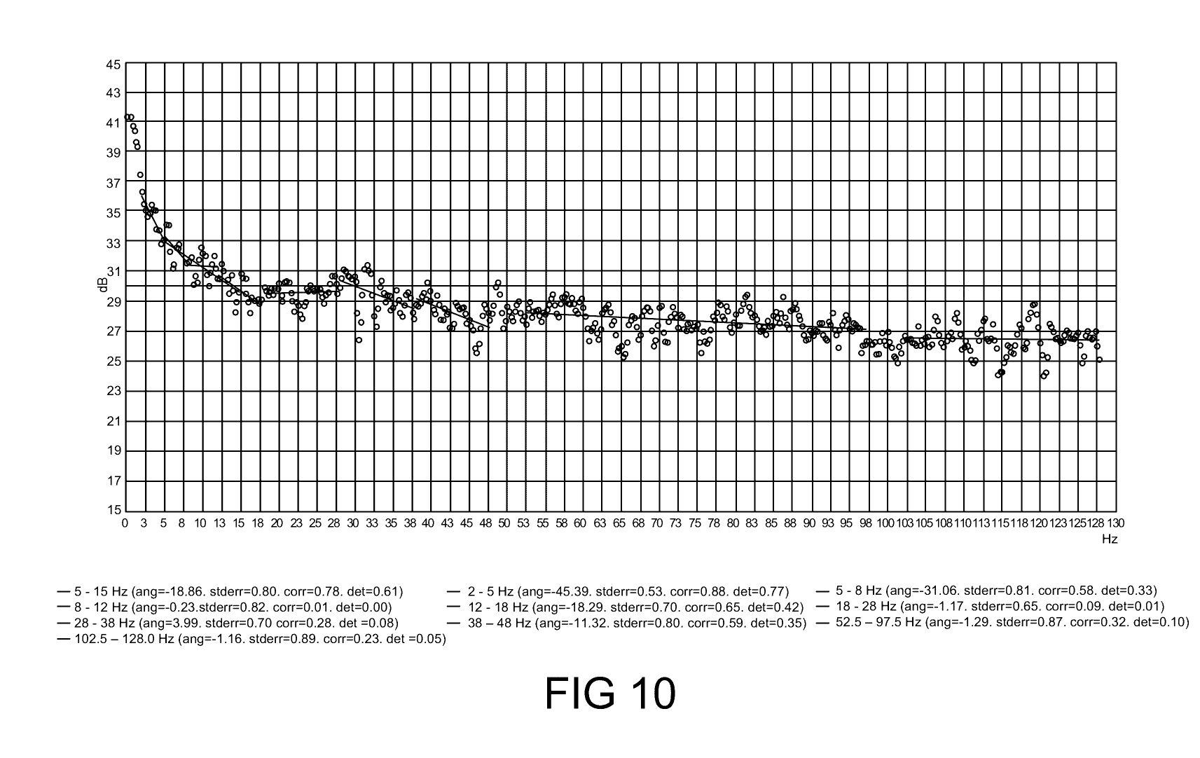

FIG. 9 and FIG. 10 show example GUI graphical demonstrations of the difference between relaxed and stress brain waves as presented with Fourier analyzed frequencies, max value frequency algorithm and regression analysis of results. FIG. 9 graph describes a relaxed brain and FIG. 10 shows a stressed brain.

The Brain Stress Index (BSI) is calculated from a combination of parameters derived from first doing a Fourier Transformation of the measurement and then doing regression analytics calculations from the recorded frequencies. The BSI use Fourier transformation to calculated frequencies ranging from 3 Hz to 128 or 256 Hz. These frequency data points are calculated from a proportion of maximum frequency values as achieved after dividing the measurement into 4 or more parts and then using the majority of maximum values for each frequency value which is at least four data points per frequency. The BSI is calculated from the variations between frequency points, the frequency value differences from point-to-point, the pattern of peak values, their peak and low value absolute strength, the angle of regression lines, the error of regression analytics and optionally BSI change from normal BSI and machine learning of individualized levels of BSI compared to measured BSI.

The Heart Frequency Stress Index (HFSI) is calculated by using in part the same logic as for the ‘Brain Stress Index’. The heart stress index utilize Fourier calculated frequencies ranging from mainly 5 Hz to 20 Hz. These frequencies are derived as a selected proportion of maximum signal values as achieved when dividing the measurement into 4 or more parts and then using the majority of maximum values achieved for each frequency value which is at least four data points per frequency. The HFSI is calculated from the variation, the frequency values differences, their absolute strength, the angle of regression lines, the error of regression analytics and optionally pulse change from normal pulse and machine learning of individualized levels of heart stress compared to measured heart stress.

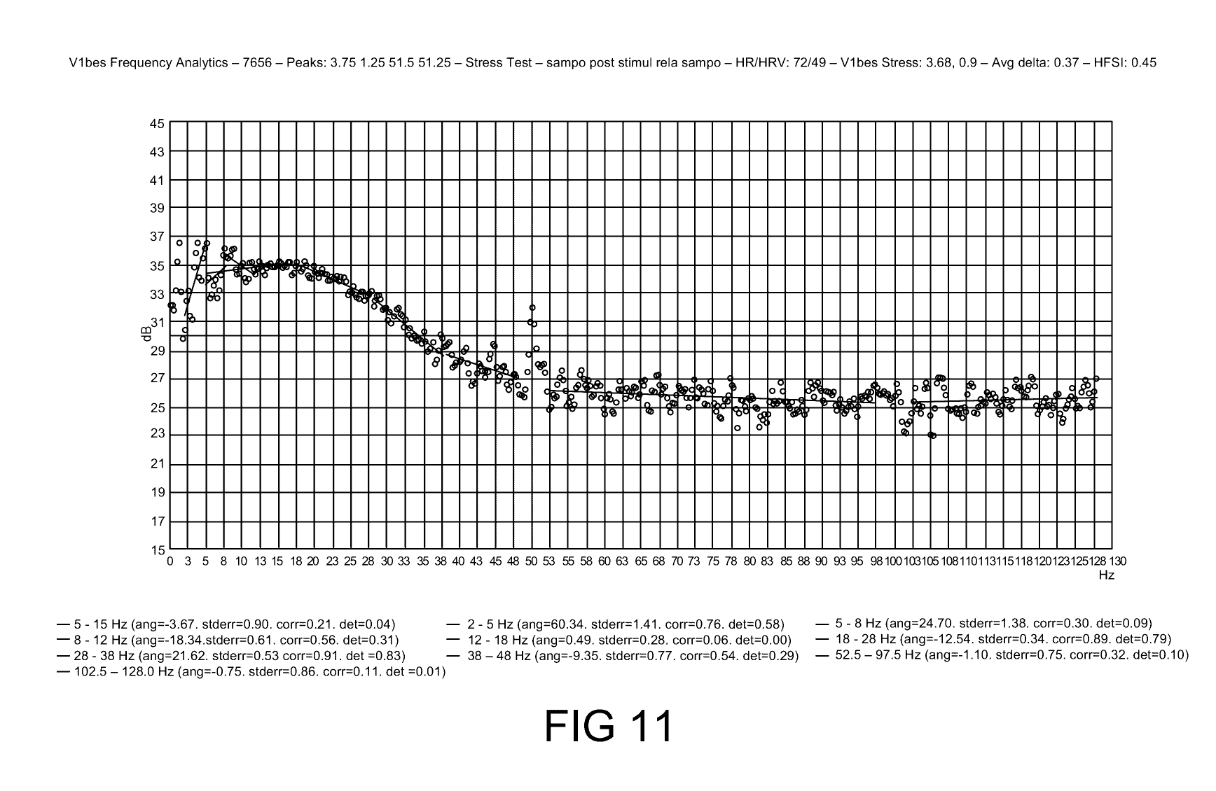

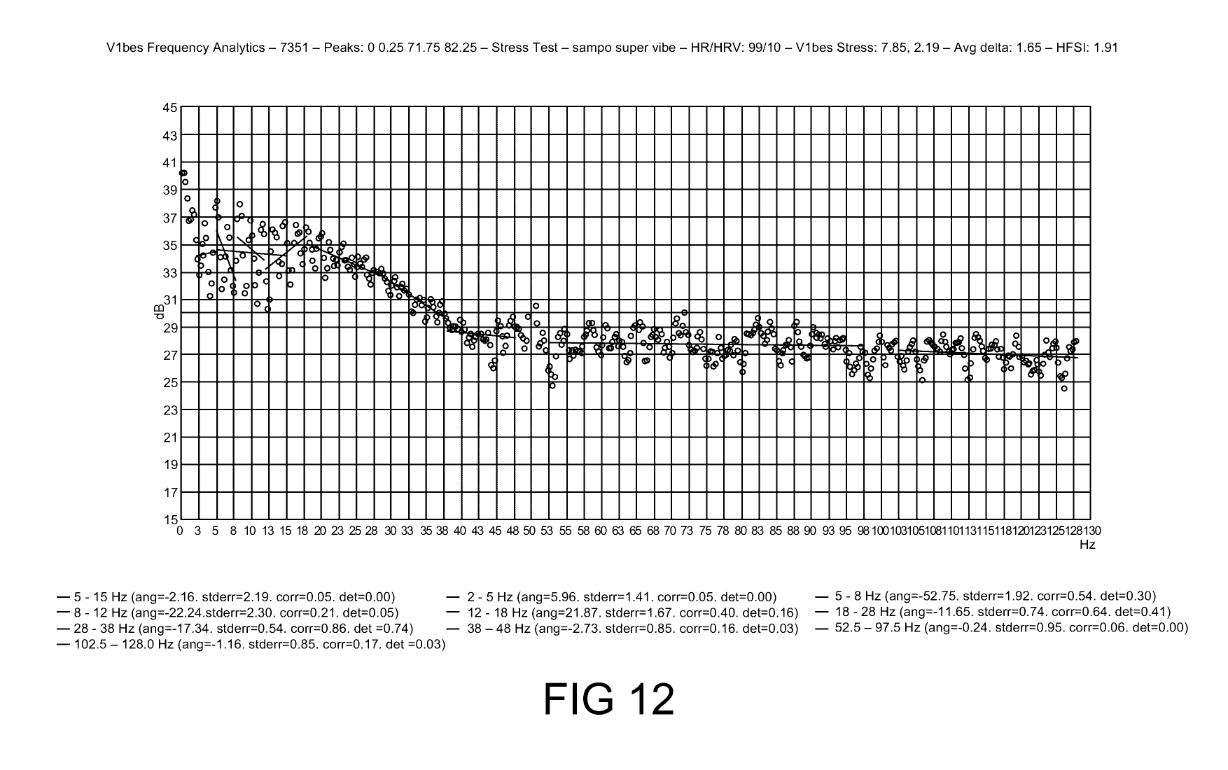

FIG. 11 and FIG. 12 shows an example GUI graphical demonstration of the difference between relaxed and stress heart as presented with Fourier analyzed frequencies, max value frequency algorithm and regression analysis of results. FIG. 11 graph describes a relaxed heart and FIG. 12 shows how a stressed heart has systematic standard deviation around regression lines at the less than 20 Hz frequency area.

### Example Hardware

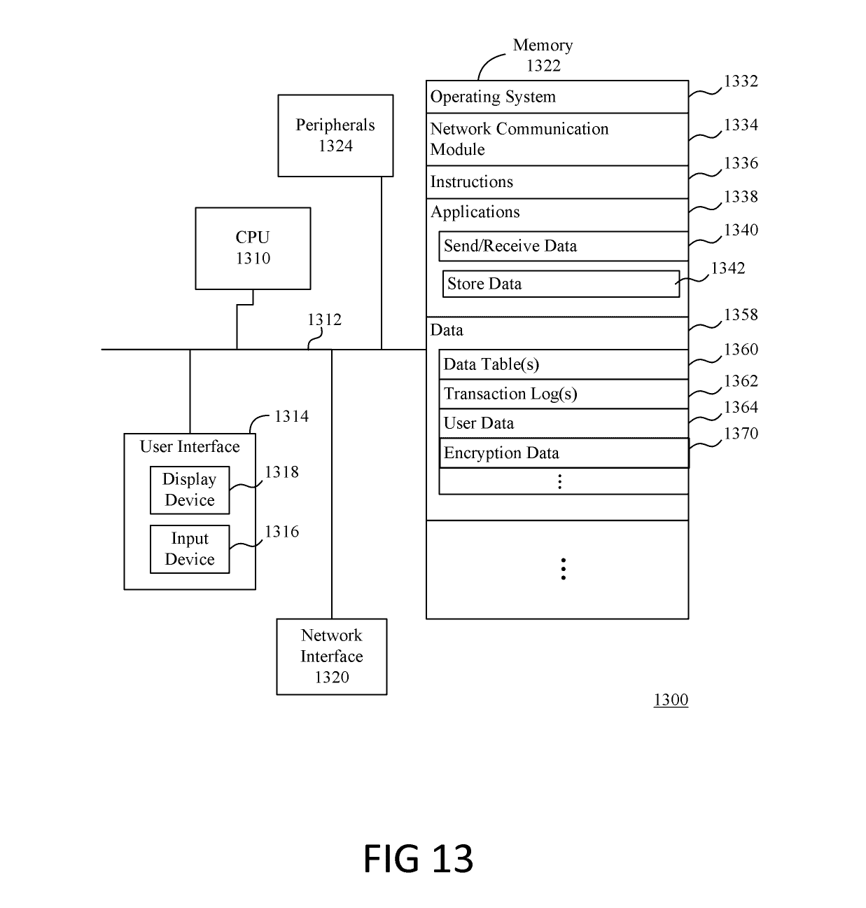

FIG. 13 shows an example computing device which may be used in practicing the various embodiments described herein. In FIG. 13, a computing device 1300 could be any of various computers including a smartphone. The computing device 1300 example here includes a processor or central processing unit 1310 in communication via a bus or other communication 1312 with a user interface 1314. In certain examples, the user interface could include a display device 1318 and input device 1316 such as a touch screen, buttons, etc. The CPU 1310 may also be in communication with a network interface 1320 which could include any of various antennae devices. Peripherals 1324 may also be used including a global positioning sensor, inertia sensors, proximity sensors, lights, LEDs and the like.

Also in communication with the CPU is a memory 1322. The memory 1322 may include software which utilizes instructions to carry out tasks. Certain software examples may include an operating system 1332, a network communication module 1334, instructions for other tasks 1336 and applications 1338 such as sending and receiving data 1340 and storing data 1342 as described herein. Data storage 1358 may also be present. Such data storage 1358 may include data tables 1360, transaction logs 1362, user data 1364 and encryption data 1370 among other data.

Such computing devices 1300 could have other arrangements, configurations, components, instructions, etc. to carry out the various tasks as described herein.

### Back End Systems and Data Storage

As described herein, the data measured by the measuring device, of the electro-magnetic signals of a user's heart and/or brain, along with in certain embodiments, answers to other biometric questions, and/or information from the social networking sites of the users, can be analyzed for various purposes. Back end computer servers may be used for this analysis. Such analysis may be used to select a data track to be played by the stimulation device, and achieve a desired goal, usually either excitement or relaxation. Certain examples allow for a range of options to be presented to a user for selection to achieve a desired result.

Certain analysis may include classifying and recognizing the recorded signals. Pattern recognition, machine learning, subjective feedback and unique stress algorithms for both brainwaves (EEG) and heart rate (ECG) may be used to determine the usability and classification of recorded biosignals. The analysis may allow social sharing and re-distribution of recorded biosignals and user generated or created stimulation programs.

In certain example embodiments, such analysis may be used to determine a neurological type. Through this data, biosignal patterns and stimulation reaction patters may be found that can be used to define a human ‘neurological type's’ of the tested persons. Such ‘neurological types’ may prefer each other's company. The systems and methods here can be used for social interaction whereby people can discover their neurological types and hence determine which neurological types they like to interact with. This matching feature could enable people to understand their partners, friends and social surrounding much better.

Data storage may be achieved by the back end systems as well. In certain examples, any of various local or network based data storage may be used to store the user data as it is collected. Such information from the measurement device, of the electrical signals of the heart and/or brain, may be stored in distributed and/or localized data storage. It may also be stored on the user's device as well or in lieu of back end storage. Such information such as trend data may also be stored as well, related to the trends that are gathered over time for a particular user or groups of users. User data such as demographics and also social network data may be used as well to group data, analyze data and chart data.

### Social Networking

In certain embodiments the smartphone application may be used to connect to the user's social networking websites and accounts. In the current western society, much emphasis is placed on the possibilities of people to share and experience through social media interaction. Much research has been done in trying to understand how and which humans best interact and improve the life other persons. The data gathered from the social networking sites of the user may be used in the analytics steps by the back end systems.

Networked storage systems and back end servers may employ algorithms used for social sharing, feedback, individual ‘Neurological Type’ classification and matching facilities enabling people to connect to ‘neurologically/or biosignal matching persons’.

Certain example embodiments may allow users to access their data from the systems here, and share that data or analytics on a social networking site, a data sharing site, or any kind of third party or proprietary website.

In certain example embodiments, if the user so wishes, the algorithms can determine the ‘type’ of the user. For example, the social networking features include using machine intelligence to determine the user's neurological type or what is here called ‘type’. The ‘type’ classification may be based on a number of pattern recognitions from the person's heart frequency, brainwave patterns and algorithms as described herein.

The reliability of a person's ‘type’ can be improved by recording with measurement device (as described in measurement device) the users biosignal reactions to sequences of standard ‘Type Testing’ (VTT) stimulation programs with stimulation device (as described in Stimulation device). These testing programs are designed to create recognizable response patterns that in turn can be used to improve the type classification.

Certain example social networking features may include allowing the users to share or post various data. Examples of information that a user may be able to share includes, but is not limited to, measurement device recordings, subjective feedback parameters as described above, subjective opinions and feedback after testing specific stimulation programs, their created stimulation programs, their neurological Types.

The systems may allow users to share either to all other users of this or then the user can send stimulation files to specifically chosen other users. The stimulation files can also be shared and sent to other persons through commonly used social media site's such as for instance Facebook, Twitter, SMS, message board, and/or email.

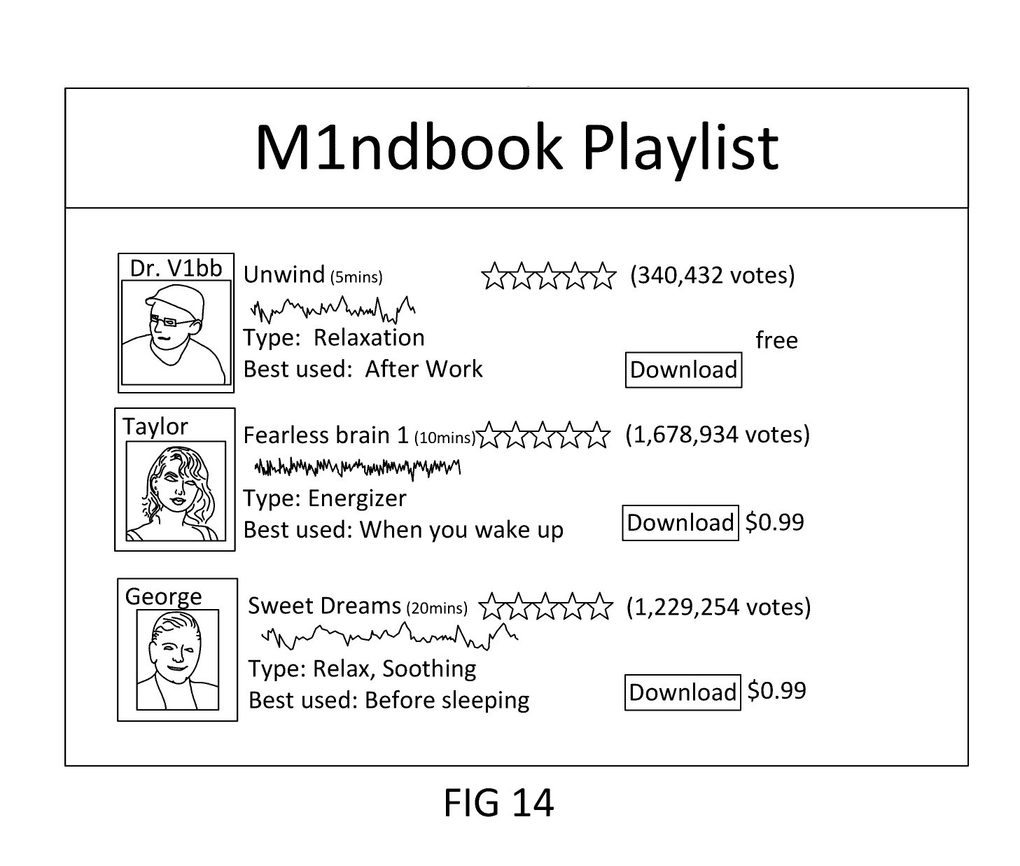

FIG. 14 shows an example of a social networking screenshot, where various celebrities have their brain waves recorded and are available for users as either stand alone and or mixed with another signal such as music for download by users.

The user can search for people with similar or otherwise matching Type's. This feature is called the Match and can be considered to be a neurological biosignal based matchmaking. Certain embodiments may employ machine learning capabilities to continuously improve automatic matchmaking algorithms.

The App, The networked data storage and systems can also collect and use other types of user data such as location data from the smartphone, music preferences or other types of individualized data in order to improve the matchmaking and type classification precision and algorithms.

### Conclusion

As disclosed herein, features consistent with the present inventions may be implemented by computer-hardware, software and/or firmware. For example, the systems and methods disclosed herein may be embodied in various forms including, for example, a data processor, such as a computer that also includes a database, digital electronic circuitry, firmware, software, computer networks, servers, or in combinations of them. Further, while some of the disclosed implementations describe specific hardware components, systems and methods consistent with the innovations herein may be implemented with any combination of hardware, software and/or firmware. Moreover, the above-noted features and other aspects and principles of the innovations herein may be implemented in various environments. Such environments and related applications may be specially constructed for performing the various routines, processes and/or operations according to the invention or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the invention, or it may be more convenient to construct a specialized apparatus or system to perform the required methods and techniques.

Aspects of the method and system described herein, such as the logic, may be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as 1PROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks by one or more data transfer protocols (e.g., HTTP, FTP, SMTP, and so on).

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

Although certain presently preferred implementations of the invention have been specifically described herein, it will be apparent to those skilled in the art to which the invention pertains that variations and modifications of the various implementations shown and described herein may be made without departing from the spirit and scope of the invention. Accordingly, it is intended that the invention be limited only to the extent required by the applicable rules of law.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

What is claimed is:

1. A system for analyzing biosignals, comprising:
a dual purpose measuring/stimulation device, in communication with a smartphone running a software application, a back end server, and a data storage,
the measuring/stimulation device configured to,
measure electromagnetic fields of a user;
send the measured electromagnetic fields to the smartphone software application;
the software application configured to,
send the received data to the back end server for processing;
receive processed data from the back end server;
send a stimulation signal to the measuring/stimulation device according to the received processed data.

2. The system of claim 1 wherein the stimulation signal is derived from recorded user electromagnetic fields.

3. The system of claim 1 wherein the stimulation uses low strength magnetic field between 0.1 and 6 micro Teslas.

4. The system of claim 1 wherein the stimulation device is stereo with two coils which work in concert.

5. The system of claim 4 wherein the measuring/stimulation device is further configured to play the stimulation signal.

6. A method for analyzing biosignals, comprising:
measuring a magnetic bio signal using a computer with a processor and memory via a measuring/stimulating device;
processing the measured bio signal at the computer;
analyzing by the computer the received measured bio signal;
choosing by the computer a stimulation signal, based on the received measured bio signal; and
playing the chosen stimulation signal via the measuring/stimulating device.

7. The method of claim 6 wherein the measuring/stimulating device includes wire coils in a headset arrangement.

8. The method of claim 6 wherein the stimulation signal includes a low strength magnetic field between 0.1 and 6 micro Teslas.

9. The method of claim 6 further comprising, causing display of the analyzed bio signal.

10. The method of claim 6 wherein the bio signal is a heart rate.

11. The method of claim 6 wherein the bio signal is a brainwave.

12. The method of claim 6 further comprising, measuring electromagnetic field of the surroundings of a user, and
wherein the analysis includes readings from electromagnetic surroundings.

13. The method of claim 6 wherein the analysis further includes user input data.

14. A non-transitory computer readable media for measuring and administering bio signals, the method comprising:
measuring at a computer with a processor and memory, a magnetic bio signal using a measuring/stimulating device;
sending by the computer the measured bio signal to a remote processing computer for processing;
receiving at the remote processing computer, the measured bio signal and processing the bio signal;
analyzing at the remote processing computer, the received measured bio signal;
choosing at the remote processing computer, a stimulation signal, based on the received measured bio signal;
sending at the remote processing computer the chosen stimulation signal to the computer;
playing, by the computer via the measuring/stimulating device the stimulation signal.

15. The non-transitory computer readable media of claim 14 wherein the measuring/stimulating device includes coiled wires.
